(19) United States
(12) Patent Application Publication
(10) Pub. No.: US 2013/0310734 A1
(43) Pub. Date: Nov. 21, 2013

(54) CATHETER FOR PHOTODYNAMIC THERAPY

(71) Applicant: Sinuwave Technologies, Inc., Bothell, WA (US)

(72) Inventors: Merrill Biel, Mendota, MN (US); Nicolas Loebel, Woodinville, WA (US); Soja-Marie Callahan Morgens, Sunnyvale, CA (US)

(73) Assignee: Sinuwave Technologies, Inc., Bothell, WA (US)

(21) Appl. No.: 13/897,936

(22) Filed: May 20, 2013

Related U.S. Application Data
(60) Provisional application No. 61/649,510, filed on May 21, 2012.

(51) Int. Cl. A61M 3/02; A61M 25/00

(52) U.S. Cl. CPC A61M 3/0283; A61M 25/0026
USPC 604/28; 604/35

(57) ABSTRACT

The present invention provides a multi-functional catheter that can be used to irrigate a treatment site located within a body cavity, to suction waste materials from the treatment site, and to deliver a treatment composition in a generally uniform distribution pattern onto the treatment site. The present invention also provides a method to use this multi-functional catheter for various applications including medical applications (e.g., photodynamic therapy treatment of chronic recurrent sinusitis).

This application claims file benefit of the filing date of U.S. Provisional Application Ser. No. 61/649,510 titled: “CATHETER FOR PHOTODYNAMIC THERAPY” filed on May 21, 2012, which is incorporated herein by reference for all purposes.

### FIELD OF INVENTION

The present invention relates to a multifunctional catheter that can be used in photodynamic therapy to irrigate a treatment site located within a body cavity (e.g., the human maxillary sinus for treatment of chronic recurrent sinusitis), to suction waste materials from the treatment site, and to deliver a treatment composition in a generally uniform distribution pattern to the treatment site.

### BACKGROUND

Chronic recurrent sinusitis (“CRS”) is an inflammatory disease of the facial sinuses and nasal passages. The National institute for Health Statistics estimates that CRS is one of the most common chronic conditions in the United States affecting an estimated 37 million Americans. See National Center for Health Statistics, NCHS, “Chronic sinusitis.” In: Summary Health Statistics for US Adults, 2002, Hyattsville, Md.: Centers for Disease Control, US Department of Health and Human Services, 2002. in clinical practice, there is a significant subpopulation of patients with CRS who remain resistant to core despite rigorous treatment regimens including surgery, allergy therapy and prolonged antibiotic therapy. The reason for treatment failure is thought to be related to the destruction of the sinus mucociliary defense by the chronic sinus infection resulting in the development of secondary antibiotic resistant microbial colonization of the sinuses and biofilm formation. Gram-negative and Gram-positive bacteria, including but not limited to, Haemophilus influenza, Streptococcus pneumonia, Staphylococcus aureus (MRSA) and multidrug resistant Pseudomonas aeruginosa are believed to cause the recalcitrant nature of persistent CRS.

Photodynamic therapy (PDT) can be used to treat various diseases involving cancer cells or microbiological pathogens. PDT is a medical treatment involving the use of a photosensitizing agent which is exposed to a specific wavelength of light to create oxygen radicals, resulting in the destruction of cancer cells, bacteria, viruses or fungi. A PDT system consists of two principal components: a photosensitizing composition and a light delivery system. The light delivery system for PDT delivers light onto the treatment site.

PDT involves the use of a photosensitizing composition that is relatively selectively concentrated in cancer cells or microbiological pathogen sites. Depending on the type of photosensitizer, the photosensitizing composition may he injected intravenously, ingested orally or applied topically. For treatment of cancer cells or microbiological pathogens within a specific body cavity area (e.g., nasal passages, maxillary and other sinuses), the photosensitizer is normally applied topically. After application, the photosensitizing composition is generally selectively retained by diseased tissue so that after a period of time, determined by the distribution kinetics of the compound, more photosensitizing composition is absorbed by the diseased tissue or microbiological pathogens than in normal tissue. The photosensitizing composition is then activated by exposure to a specific wavelength of light matching the absorption spectrum peak of the composition. This results in oxygen-derived free radical production and consequent tissue necrosis via several mechanisms including vascular constriction to the diseased tissue. Because there is less photosensitizing composition in the adjacent normal tissue, only the diseased tissue and/or microbiological pathogens undergo necrosis and the normal tissue is preserved when the correct light dose rate for that tissue is administered. The advantage of PDT over conventional treatment such as surgery, radiation and chemotherapy is its relatively selective destruction of diseased tissue or microbiological pathogens with normal tissue-preservation.

Using PDT to treat CRS requires delivery of the photosensitizing composition to the treatment site such as the maxillary sinuses which can be accomplished by using a catheter. The distribution of the photosensitizing composition onto the treatment site can have direct impact on the efficacy of the PDT treatment. PDT treatment is more effective when the photosensitizing composition is delivered by the catheter in a generally uniform distribution pattern onto the treatment site because it allows the PDT light activation of the photosensitizer to be generally uniform within the treatment site (hereinafter referred to as “generally uniform distribution pattern”). If the catheter does not provide a generally uniform distribution pattern of the photosensitizing composition within the treatment site, the treatment site may receive too much, too little or an uneven distribution of the photosensitizing composition which in torn is likely to lower the PDT efficacy.

Furthermore, it is generally desired that the treatment site be cleaned of debris or waste materials before the application of the photosensitizing composition onto the treatment site. Removal of waste materials allows the photosensitizing composition to have a better interface with the targeted cancer ceils or microbiological pathogens located at the treatment site. Accordingly, a catheter that can irrigate the treatment site with biocompatible fluids (e.g., saline, water, or the like) and aspirate the biocompatible fluids along with any waste materials located within the treatment site (hereinafter respectively referred to as “irrigation” and “aspiration”) is desired to be used before application of the photosensitizing composition onto the treatment site.

### SUMMARY OF INVENTION

The present invention provides an improved and multifunctional catheter that provides irrigation, aspiration, arid delivery of a treatment composition (e.g., a photosensitizing composition, or other medicine) in a generally uniform distribution pattern to a treatment site located within a body cavity. This catheter is useful for many applications including but not limited to PDT treatments of desired treatment site(s) located within body cavities such as the maxillary sinuses.

The catheter of the present invention comprises a handle hub, a first tube, a second tube, a control device, a connector, and a delivery assembly comprising a third tube having a third lumen and delivery nozzles, a metal tube having a metal lumen, a fourth tube having a fourth lumen, and a delivery tip having a lip lumen. The delivery assembly includes a double lumen structure with an outer lumen and an inner lumen that is coaxial with the outer lumen and located within the outer lumen. The volume of the outer lumen is less than the volume of the inner lumen. The metal tube and the fourth tube are located within the third lumen and the outer lumen is formed by space remaining in the third lumen not occupied by the metal tube, the metal lumen, the fourth tube and the fourth lumen. The inner lumen is comprised of at least a portion of the metal lumen, at least a portion of the fourth tube, and at least a portion of the tip lumen. The distal end of the first tube is connected to the handle hub and proximate end of the first tube is connected to the connector. The connector is removably connected to a container containing a treatment composition. The distal end of the second tube is connected to the handle hub and proximate end of the second tube is connected to the control device. The control device is removably connected to a vacuum device and an irrigation fluid supply. The delivery assembly is connected to the handle hub. The control device controls irrigation and aspiration by the inner lumen of the delivery assembly when the control device is connected to the vacuum device and the irrigation fluid supply. The delivery nozzles are in communication with the outer lumen so when the connector is connected to the container, the delivery nozzles can deliver the treatment composition in a generally uniform distribution pattern to a treatment site.

The present invention also provides a method to use this multi-functional catheter comprising providing the catheter of the present invention described above; connecting the control device to the aspiration line of the vacuum device; and connecting the control device to the irrigation line of the irrigation fluid supply. The method further includes inserting the delivery assembly info a body cavity until distal portion of the delivery assembly is located at a predetermined location near a treatment site; irrigating the treatment site using the catheter wherein the control device controls the amount of irrigation fluid being delivered by the delivery assembly out of the delivery tip; and aspirating the treatment site to remove waste material using the catheter wherein the control device controls the amount of suction being delivered by the delivery assembly out of the delivery tip. The method also includes applying a treatment composition in a generally uniform distribution pattern onto a treatment site using the catheter when the connector is connected to the container and the delivery nozzles are in liquid communication with the container. Finally, the method includes removing the delivery assembly from the body cavity.

### BRIEF DESCRIPTION OF THE DRAWINGS

The features and inventive aspects of the present invention will become more apparent upon reading the following detailed description, claims, and drawings, of which the following is a brief description:

FIG. 1 is a plan view of plan exemplary embodiment of a catheter of the present invention;

FIG. 2 is an enlarged plan view of the distal portion of a delivery assembly of the catheter shown in FIG. 1;

FIG. 3 is an enlarged plan view of a handle hub of the catheter shown in FIG. 1;

FIG. 4 is an enlarged side view of the handle hub shown in FIG. 3;

FIG. 5 is a cross sectional view of the handle hub shown in FIG. 3 connected to a first tube, a second tube and proximate end of the delivery assembly as shown in FIG. 1;

FIG. 6 is a cross sectional view of the distal portion of the delivery assembly shown in FIG. 2;

FIG. 7 is a sectional view taken on line 41-41 of FIG. 6;

FIG. 8 is a sectional view taken on line 43-43 of FIG. 6;

FIG. 9 is a sectional view taken on line 45-45 of FIG. 6;

FIG. 10 is an enlarged plan view of a control device of the catheter shown in FIG. 1 connected to an aspiration line of a suction or vacuum device and an irrigation line of an irrigation fluid supply;

FIG. 11 is an enlarged plan view of a control device of the catheter shown in FIG. 1 connected to an irrigation line of an irrigation fluid supply; and

FIG. 12 is an enlarged plan view of the control device shown in FIG. 11 connected to an aspiration line of a suction or vacuum device.

### DETAILED DESCRIPTION OF THE INVENTION

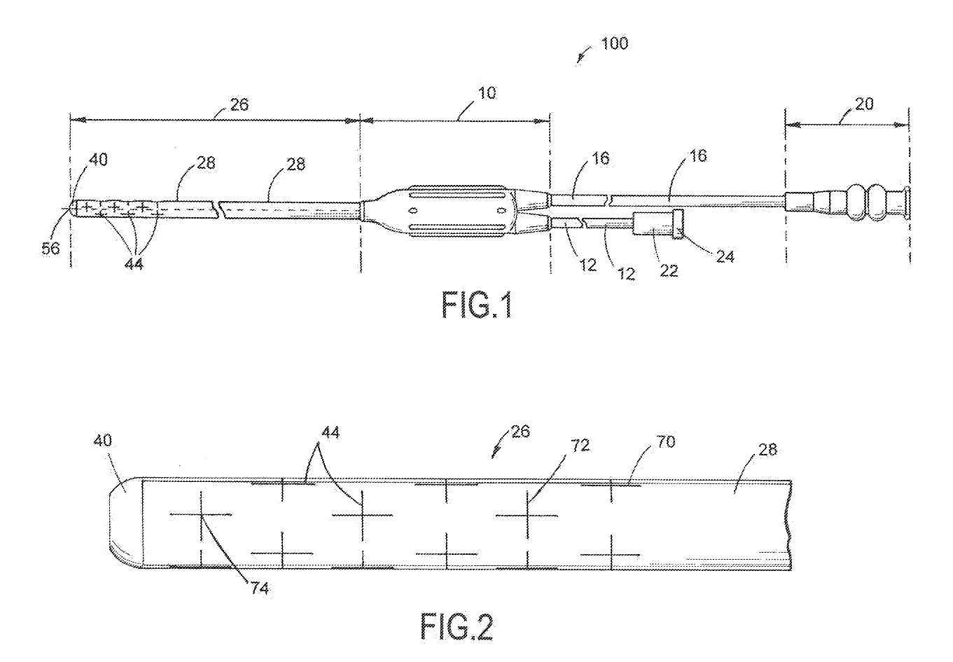

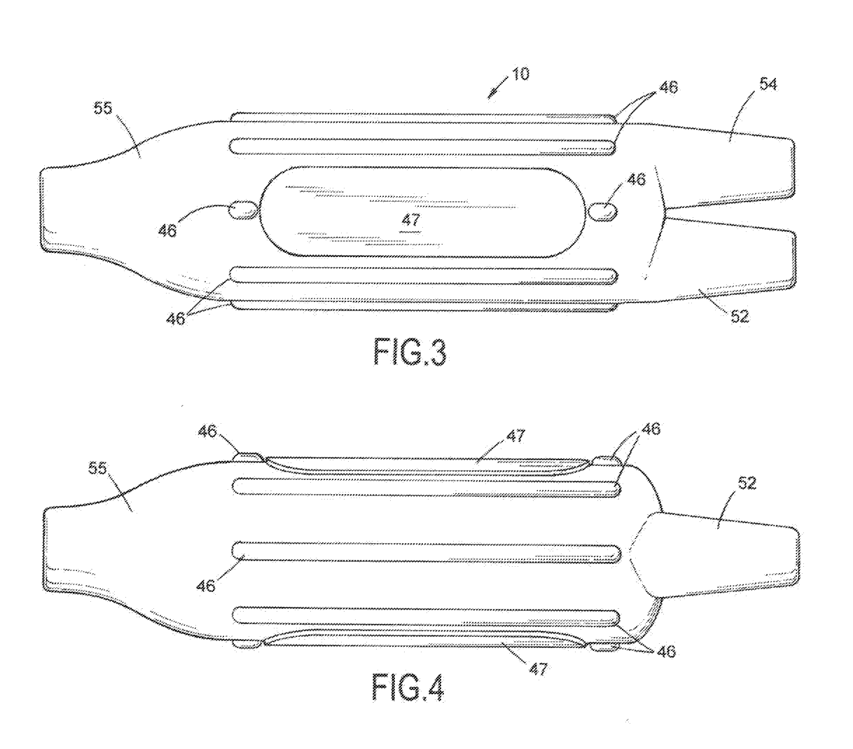

FIG. 1 illustrates one exemplary embodiment of the improved and multifunctional catheter 100 of the present invention. The catheter 100 provides irrigation, aspiration, and delivery of treatment composition in a generally uniform distribution pattern to a treatment site. Referring to FIGS. 1-10, the catheter 100 comprises a handle hub 10, a first tube 12 having a first lumen 14, a second tube 16 having a second lumen 13, a control device 20 for controlling irrigation and aspiration, a connector 22 with connector features 24, a delivery assembly 26 comprising a third tube 28 having a third lumen 30, a metal tube 32 having a metal lumen 34, a fourth tube 36 having a fourth lumen 38, a delivery tip 40 having a tip lumen 42, and delivery nozzles 44.

Referring to FIGS. 1 and 3-5, the handle hub 10 has a generally cylindrical shape constructed out of art-disclosed suitable material(s) such as plastic, polymer or the like. For example and in one exemplary embodiment, the handle hub 10 is constructed out of a thermoplastic material (e.g., Pellethane®). Grip features 46 are optionally provided on the exterior surface of the handle hub 10 in order to provide the user with better handling of the catheter 100. Label features 47 are also optionally provided on the exterior surface of the handle hub 10 in order to provide a desired location for written information (e.g., product name, trademarks patent pending disclosure, or the like). A first hub lumen 48 and a second hub lumen 50 are included and located within the handle hub 10. The handle hub 10 further includes a first proximate port 52, a second proximate port 54, and a distal port 55.

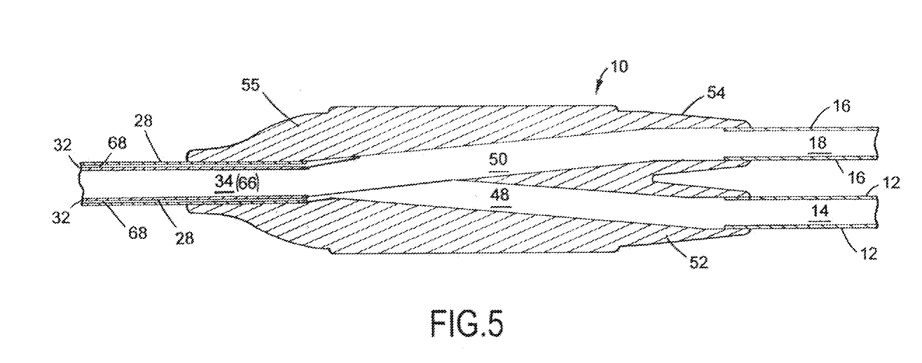

Referring to FIG. 5, the distal end of the first tube 12 is connected to a first proximate port 52 of the handle hub 10 providing the proximate end of the first hub lumen 48 with the ability to be in liquid communication with the distal end of the first lumen 14 located within the first proximate port 52. The distal end of the second tube 16 is connected to a second proximate port 54 of the handle hub 10 providing the proximate end of the second hub lumen 50 with the ability to be in fluid and suction communication with the distal end of the second lumen 18 located within the second proximate port 54.

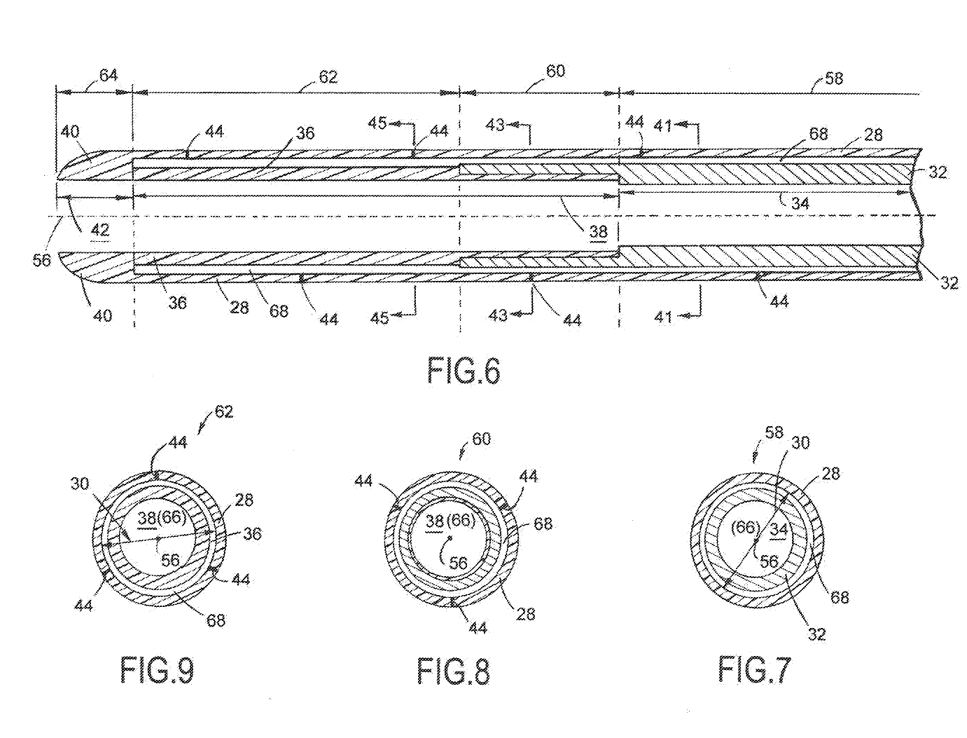

Referring to FIGS. 6-9, the delivery assembly 26 includes a central axis 56 and has four sections: a first assembly section 58, a second assembly section 60, a third assembly section 62, and a fourth assembly section 64. FIG. 7 shows a sectional view of the first assembly section 58 taken at line 41-41 shown in FIG. 6. FIG. 8 shows a sectional view of the second assembly section 60 taken at line 43-43 shown in FIG. 6. FIG. 9 shows a sectional view of the third assembly section 62 taken at line 45-45 shown in FIG. 6. Each of the four sections (58, 60, 62, and 64) includes an inner lumen 66 that extends along the central axis 56 from the proximate end of the delivery assembly 26 to the distal end of the delivery assembly 26. In the first assembly section 58, the inner lumen 66 is the metal lumen 34 as shown in FIG. 7. In the second assembly section 60 and the third assembly section 62, the inner lumen 66 is the fourth lumen 38 as shown in FIGS. 8-9. In the fourth assembly section 64, the inner lumen 66 is the tip lumen 42 as shown in FIG. 6.

Referring to FIGS. 6-9, the first assembly section 58, the second assembly section 60, and the third assembly section 62 of the delivery assembly 26 all comprise a double lumen structure with the inner lumen 66 and an outer lumen 68. The outer lumen 68 extends along the central axis 56 from the proximate end of the delivery assembly 26 to the distal end of the third assembly section 62.

In one exemplary embodiment, the third tube 28, the metal tube 32, and the fourth tube 36 are all coaxial and have the central axis 56 as their radical axis so that they are concentric to one another as shown in FIGS. 6-9. It is optional that the third tube 28, the metal tube 32, and the fourth tube 36 are coaxially symmetric.

Referring to FIGS. 6-7 and in the first assembly section 58, the third tube 28 and the metal tube 32 form the double lumen structure wherein the metal tube 32 resides within the third lumen 30 leaving the metal lumen 34 with a diameter lesser than the diameter of the third lumen 30. The metal lumen 34 is the inner lumen 66 and the volume (e.g., space or area of the third lumen 30 not occupied by (e.g., outside of) the metal tube 32 and the metal lumen 34 forms the portion of the outer lumen 68 that is within the first assembly section 58.

Referring to FIG. 5, the proximate end of the metal tube 32 is connected to the distal port 55 of the handle hub 10 providing the proximate end of the metal lumen 34 (and the inner lumen 66) with the ability to be in fluid and suction communication with the distal end of the second hub lumen 50. The proximate end of the third tube 28 is also connected to the distal port 55 providing the proximate end of the outer lumen 68 with the ability to be in liquid communication with the distal end of the first hub lumen 48.

In the second assembly section 60 and referring to FIGS. 6 and 8, the distal end of the metal tube 32 is connected to the proximate end of the fourth tube 36 providing the proximate end of the fourth lumen 38 with the ability to be in fluid and suction communication with the distal end of the metal lumen 34. The diameter of the metal lumen 34 within the second assembly section 60 is larger than the diameter of the metal lumen 34 within the first assembly section 58 so that it can accommodate the proximate end of the fourth tube 36. Suitable art-disclosed adhesive can fee optionally use to assist in the connection between the distal end of the metal tube 32 and the proximate end of the fourth tube 36. In the second assembly section 60, the inner lumen 66 is the fourth lumen 38, and the outer lumen 68 is formed by the interior wall of the third tube 28 (e.g., the surface wall of the third lumen 30) and the exterior wall of the metal tube 32.

Instead of having the proximate end of the fourth tube 36 located inside the metal lumen 34 as described above, in an alternative exemplary embodiment, the diameter of the fourth lumen 38 within the second assembly section 60 is larger than the outer diameter of the metal tube 32 so that the fourth lumen 38 within the second assembly section 60 can accommodate the distal end of the metal tube 32. Suitable art-disclosed adhesive can be optionally use to assist in the connection between the distal end of the metal tube 32 and the proximate end of the fourth tube 36. For this embodiment, the second, assembly section 60, the inner lumen 66 is the metal lumen 34 and the outer lumen 68 is formed by the interior wail of the third tube 28 (e.g., the surface wall of the third lumen 30) and the exterior wall of the fourth tube 36.

All of the connections and/or attachments discussed herein for the various components such as tubes (e.g., 12, 16, 28, 32 and 36), ports (e.g., 52, 54 and 55), connector (22), control device (20), and tip (40) can be achieved using suitable art-disclosed means, including but hot limited to, the methods described above for the connection between the distal end of the metal tube 32 and the proximate end of the fourth tube 36.

In the third assembly section 62 and referring to FIG. 6 and 9, the third tube 28 and the fourth tube 36 form the double lumen structure wherein the fourth tube 36 resides within the third lumen 30 leaving the fourth lumen 38 with a diameter lesser than the diameter of the third lumen 30. The fourth lumen 38 is the inner lumen 66 and the area of the third lumen 30 outside of the fourth tube 36 and the fourth lumen 38 forms the portion of the outer lumen 68 that is within the third assembly section 62. The fourth tube 36 and the outer lumen 68 both terminate within the third assembly section 62 as shown in FIG. 6. The distal end of the fourth tube 36 is located at a suitable, location within the third assembly section 62 that provides the distal end of the fourth lumen 38 with the ability to be in fluid and suction communication with the proximate end of the tip lumen 42.

Referring to FIG. 6 and in the fourth assembly section 64, the double lumen structure of the third assembly section 62 described above no longer exists. The only lumen remaining in the fourth assembly section 64 is the tip lumen 42 forming the inner lumen 66. When the catheter 100 is in operation, the distal end of the tip lumen 42 provides irrigation and/or aspiration to the treatment site.

In one embodiment, the delivery tip 40 is a separate component attached to the other components of the fluid assembly 26 (e.g., the third tube 28 and the fourth tube 36). Alternatively, the delivery tip 40 and the third tube 28 are formed or constructed as a single component wherein the third lumen 30 and the tip lumen 42 are the same seamless structure,

If is optionally provided that the delivery assembly 26 is atraumatic. To assist in making the delivery assembly 26 atraumatic, the delivery tip 40 has a smooth exterior surface (e.g., each edge of the delivery tip 40 is radiused). The third assembly section 62 and the fourth assembly section 64 are constructed of suitable materials) that make them atraumatic. For example, the delivery tip 40 and the third tube 28 located within the third assembly section 62 and the fourth assembly section 64 are constructed of a suitable bendable and flexible material such as plastic, polymer, or the like. Furthermore, it is optional that the delivery tip 40 is constructed of a material that is softer than the material used for the construction of the third tube 28.

The atraumatic quality of the catheter 100 is assisted by having the metal tube 32 terminated at the second assembly section 60. The metal tube 32 provides stiffness and support to the delivery assembly 26 while allowing the delivery assembly 26 and itself (32) to maintain the ability to be flexible, bendable, and malleable. The metal tube 32 is constructed of a metal alloy in a desired thickness that provides all of these desired characteristics.

In order to maintain desired stiffness and support for the delivery assembly 26 and still allowing the delivery assembly 26 to be atraumatic, the combined length of the third assembly section 62 and the fourth assembly section 64 should be less than the combined length of the first assembly section 58 and the second assembly section 60. In fact, the combined length of the third assembly section 62 and the fourth assembly section 64 is likely to be substantially less than the length of the first assembly section 58. In one exemplary embodiment, the length of the fourth assembly section 64 ranges from about 0.08″, between about 0.05″ to about 0.2″, and between about 0.06″ and 0.15″; and the length of the third assembly section 62 ranges from about 0.5″, between about 0.3″ to about 0.8″, and between about 0.4″ to about 1″. Length as discussed herein in this specification is measured in the same direction as indicated in FIG. 6 for the central axis 56.

The first tube 12, the second tube 16, the third tube 28, the fourth tube 36, and the deliver) tip 40 are constructed out of suitable flexible and bendable materials) such as plastic, polymers, or the like. For example, in one exemplary embodiment, the first tube 12 and the second tube 16 are constructed out of nylon while the third tube 28, the fourth tube 36, and the delivery tip 40 are all constructed out of thermoplastic material(s) (e.g., Pebax® or the like) with similar or different durometer values.

Referring to FIG. 1, the connector features 24 allows the connector 22 to be removably connected to a liquid container (not shown) containing the treatment composition. The connector 22 is also connected to the proximate end of the first tube 12. The connector features 24 can be any art-disclosed suitable features that provide a removable but secured attachment with another component. In one exemplary embodiment, the connector features 24 are Luer-lock fillings that can be removably connected to a syringe (e.g., the fluid container) with corresponding Luer-lock fittings thereby allowing the first tube 12 to be in liquid communication with the syringe.

Referring to FIGS. 1-2 and 5-6, once the connector 22 is connected to the liquid container containing the treatment composition, a user can pump the treatment composition from the liquid container to the delivery nozzles 44 via the first tube 12, the first hub lumen 48, and the outer lumen 68. Unlike some conventional systems and methods, this liquid delivery system does not need to involve the use of gas pressure assist. To assist in the delivery of the treatment composition in the generally uniform distribution pattern onto the treatment site, the outer lumen 68 has a volume that is lower than the volume provided by the inner lumen 66 as shown in FIG. 6.

Referring to FIGS. 1-2, the delivery nozzles 44 are included in the distal portion of the third tube 28 in a specific design so that they can deliver the treatment composition in small liquid droplets (neither a fine aerosol nor a solid liquid stream, and without assistance of gas pressure) to the treatment site in a generally uniform distribution pattern. The delivery nozzles 44 are created by cuts into the distal portion of the third tube 28. The distal portion of the third tube 28 includes rows of delivery nozzles 44 with several delivery nozzles 44 in each row. Each of the delivery nozzles 44 includes a horizontal cut 70 and a vertical cut 72. The two cuts (70, 72) are perpendicular to each other it is optional that the two cuts (70, 72) are centered relative to each other when internal pressure and the treatment composition are provided within the outside lumen 66, the edges of the cuts (70, 72) open up and the shear over the edges of the cuts (70, 72) causes delivery of the treatment composition by the delivery nozzles 44 onto the treatment site in a generally uniform distribution pattern. Each of the delivery nozzles 44 in each row are rotationally set apart from each other in a predetermined amount. Furthermore, each of the delivery nozzles 44 in each row is located in an offset fashion from the delivery nozzles 44 in the adjacent row. This design and distribution of delivery nozzles 44 assist in delivering the treatment composition in small liquid droplets (e.g., droplet size ranges from about 30 μm, from about 5 μm to about 125 μm, from about 10 μm to about 100 μm, from about 15 μm to about 50 μm) in a generally uniform distribution pattern onto the treatment site.

Referring to FIG. 1, the proximate end of the second tube 16 is connected to the control device 20 providing the proximate end of the second lumen 18 with the ability to be in fluid and suction communication with the control device 20. This connection also provides the inner lumen 66 of the delivery assembly 26 with the ability to be in fluid and suction communication with the control device via the second hub lumen 50 and the second lumen 18.

Referring to FIG. 10, the control device 20 includes art-disclosed lockable features (not shown) that allow the control device 20 to be removably connected to both an aspiration line 76 of a conventional suction or vacuum device (not shown) and an irrigation line 78 of an irrigation fluid supply (not shown). The control device 20 controls the switching between aspiration and irrigation allowing the second lumen 18 (and by extension the inner lumen 66) to have the ability for bi-directional fluid flow in an effective and quick manner desired for irrigation end aspiration without having to disconnect and/or reconnect the aspiration line 76 and/or the irrigation line 78. The control device 20 can be any suitable art-disclosed control device. For an exemplary control device, please see U.S. Pat. No. 7,182,746. If desired, the catheter 100 as described herein can optionally provide a high pressure but low volume irrigation to the treatment site.

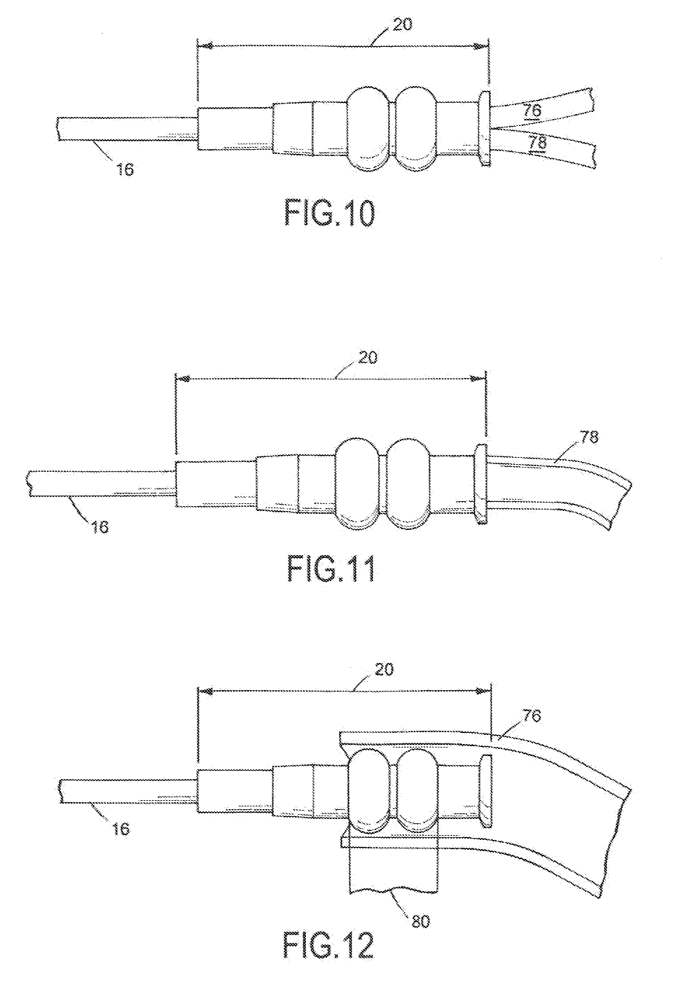

In an alternative exemplary embodiment, the control device 20 includes art-disclosed lockable features (not shown) that allows the control device 20 to be removably connected to the irrigation line 78 as shown in FIG. 11 when irrigation is desired. The control device 20 further includes a suction lock feature 80 as shown in FIG. 12. When suction is desired, the irrigation line 78 is first disconnected with the control device 20 (if applicable). Thereafter, the aspiration line 76 is removably connected to the control device 20 when distal end of the aspiration line 76 receives the control device 20 up to distal end of the suction lock feature 80 as shown in FIG. 12. In another embodiment, the suction lock feature 80 is a Luer lock feature and the distal end of the aspiration line 76 includes a corresponding Luer Lock feature that connects with the suction lock feature 80 to form a Luer connection.

In one exemplary embodiment of the catheter 100 used for PDT treatment of CRS and referring to FIGS. 1-10, the handle hob 10 is constructed of Pellethane®. The connector 22 and the irrigation and aspiration control device 20 are constructed of polyvinyl chloride (“PVC”). The connector features 24 are female Luer-lock fittings. The first, tube 12 and the second tube 16 are constructed of nylon or other suitable flexible polymeric material and both have an outer diameter of 0.09″ and a diameter of 0.07″ for the first lumen 14 and the second lumen 1$. The third tube 28, the fourth tube 36. and the delivery tip 40 are all constructed of Pebax® or other suitable flexible polymeric material. The third tube 28 has an outer diameter of 0.16″. The third lumen 30 has a diameter of 0.14″. The fourth tube 36 within the third assembly section 62 has an outer diameter of 0.125″. The fourth tube 36 within the second assembly section 60 has an outer diameter that is less than 0.125″ that can enter into the metal lumen 34 within the second assembly 60. The fourth lumen 38 has a diameter of 0.09″. The tip lumen 42 also has a diameter of 0.09″. The metal tube 32 is constructed of a malleable metal alloy and has an outer diameter of 0.125″. The metal lumen 34 within the first assembly section 58 has a diameter of 0.09″. Accordingly, the inner lumen 66 has a diameter of 0.09″ along its entire length. Subtracting the outer diameter of either the metal tube 32 or the fourth tube 36 at 0.125″ from the diameter of the third lumen 30 at 0.14″ yields one of the dimensions for the outer lumen 68 which is consistent along the entire length of the outer lumen 68.

The metal lumen 34 within the second assembly section 60 has a diameter that is greater than the outer diameter of the fourth tube 36 within the second assembly section 60. The length of each of the components within the second assembly section 60 is 0.2″. The length of the fourth assembly section 64 is 0.09″. The length of the third assembly section 62 is 0.41″.

The distal portion of the third tube 28 includes six rows of delivery nozzles 44 with three delivery nozzles 44 in each row. Each row of the delivery nozzles 44 is spaced 0.06″ apart from the next row of delivery nozzles 44. Each of the delivery nozzles 44 within each row is rotationally 60° apart as measured from the central axis 60. The horizontal cut 70 of each of the delivery nozzles 44 is 0.11″ in length and ranges from 0.009″ to 0.012″ in width. The vertical cut 72 of each of the delivery nozzles 44 is 0.08″ in height and ranges from 0.009″ to 0.012″ in width. The horizontal cut 70 and the vertical cut 72 are centered and perpendicular in respect to each other. Measuring from the center point 74 of one of the delivery nozzles 44 to the center point 74 of its adjacent delivery nozzles 44 within the same row yield a distance of 0.28″. The delivery nozzles 44 in each row do not form a straight line with the delivery nozzles 44 located in the adjacent row instead, each delivery nozzle 44 is offset by 0.14″ (based upon measurement between the two center points 74) from each of its nearby delivery nozzle 44 located in the adjacent row. This design of the delivery nozzles 44 on the distal portion of the third tube 28 assist in delivering the treatment composition (e.g., the photosensitizing composition) in small liquid droplets (not aerosol and without assistance of gas) to the treatment site in a generally uniform distribution pattern.

The dimensions and measurements provided above for the exemplary embodiment of the catheter 100 used for PDT treatment of CRS are not intended as being exhaustive or limiting of the invention. Those skilled in the art may change the dimensions and measurements of the catheter 100 as may be best suited to the requirements of a particular use and still be within the scope of the present invention.

The present invention also provides a method of using the catheter 100 for PDT treatment of CRS The method includes providing the catheter 100 of the present invention; connecting the control device 20 to the aspiration line 76 of the vacuum device; connecting the control device 20 to the irrigation line 78 of the irrigation fluid supply; inserting the delivery assembly 26 into a body cavity (e.g., the human maxillary sinus) until the delivery tip 40 is located at a predetermined location near a treatment site; irrigating the treatment site using the catheter 100 wherein the control device 20 controls the amount of irrigation fluid being delivered by the delivery assembly 26 out of the delivery tip 40; aspirating the treatment site to remove waste material using the catheter 100 wherein the control device 20 controls the amount of suction being delivered by the delivery assembly 26 out of the delivery tip 40; applying the treatment composition in a generally uniform distribution pattern onto a treatment site using the catheter 100 when the connector 22 is connected to the container and the delivery nozzles 44 are in liquid communication with the container. Finally, the method includes removing the delivery assembly 26 from the body cavity.

The method of the present invention described above may be changed to first perform the aspiration step: follow by the irrigation step, and then another aspiration step, in fact, the aspiration and irrigation steps can be each repeated as desired during treatment.

The method of the present invention described above and the catheter 100 can also be used for other medical applications other than PDT and may be modified accordingly. Moreover, they can also be used for delivery of a desired composition to other body cavities or even inanimate cavities. For example, the catheter 100 can be sized suitably (decreasing or increasing in size) to provide a desired fit within the body cavities such as ear, vagina, lung, the entire digestive tract (e.g., throat, esophagus, stomach, intestines, rectum, or the like). In one exemplary embodiment, components of the catheter 100 described above for PDT treatment of CRS are ail proportionally reduced by 30% to 50% so the catheter 100 can be used for PDT treatment in the ear cavity.

It is understood that the present invention as described and claimed herein can be used for many additional purposes, therefore the invention is within the scope of other fields and uses and not so limited.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes.

What is claimed is:

1. A multi-functional catheter comprising: a handle hub, a first tube, a second tube, a control device, a connector, and a delivery assembly comprising a third tube having a third lumen and delivery nozzles, a metal tube having a metal lumen, a fourth tube having a fourth lumen, and a delivery tip having a tip lumen; wherein
a. the delivery assembly includes a double lumen structure with an outer lumen and an inner lumen that is coaxial with the outer lumen and located within the outer lumen;
b. volume of the outer lumen is less than volume of the inner lumen;
c. the metal tube and the fourth tube are located within the third lumen and the outer lumen is formed by space remaining in the third lumen not occupied by the metal tube, the metal lumen, the fourth tube and the fourth lumen;
d. the inner lumen is comprised of at least a portion of the metal lumen, at least a portion of the fourth tube, and at least a portion of the tip lumen;
e. distal end of the first tube is connected to the handle hub and proximate end of the first tube is connected to the connector; the connector is removably connected to a container containing a treatment composition;
f. distal end of the second tube is connected to the handle hub and proximate end of the second tube is connected to the control device;
g. the control device is removably connected to a vacuum device and an irrigation fluid supply;
h. the delivery assembly is connected to the handle hub;
i. the control device controls irrigation and aspiration by the inner lumen of the delivery assembly when the control device is connected to the vacuum device and the irrigation fluid supply;
j. the delivery nozzles are in communication with the outer lumen so when the connector is connected to the container, the delivery nozzles can deliver the treatment composition in a generally uniform distribution pattern onto a treatment site; and
k. the delivery nozzles are located in distal portion of the third tube and constructed to assist in delivering the treatment composition in a generally uniform distribution pattern onto the treatment site,

2. The catheter of claim 1 wherein:
a. the delivery assembly includes a central axis;
b. the delivery assembly has a first assembly section, a second assembly section, a third assembly section, and a fourth assembly section;
c. each of the first assembly section, the second assembly section, and the third assembly section includes the double lumen structure and the fourth assembly section includes the inner lumen;
d. the outer lumen extends along the central axis from proximate end of the delivery assembly to distal end of the third assembly section;
e. in the first assembly section, the third tube and the metal tube form the double lumen structure;
f. in the second assembly section, the third tube, the metal tube, and the fourth tube form the double lumen structure;
g. the metal tube is connected to the fourth tube allowing the metal lumen to be in fluid and suction communication with the fourth lumen;
h. in the third assembly section, the third tube and the fourth tube form the double lumen structure and the inner lumen is the fourth lumen; and
i. the fourth tube is connected to the delivery tip allowing the fourth lumen to be in fluid and suction communication with the tip lumen; and
j. the fourth assembly section includes the delivery tip and the tip lumen is the inner lumen.

3. The catheter of claim 2 wherein in the second assembly section, the inner lumen is the metal lumen.

4. The catheter of claim 2 wherein in the second assembly section, the inner lumen is the fourth lumen.

5. The catheter of claim 2 wherein combined length of the third assembly section and the fourth assembly section is less than length of the first assembly section.

6. The catheter of claim 5 wherein length of the fourth assembly section ranges between 0.05 inches to about 0.2 inches and length of tie third assembly section 62 ranges between 0.3″ to about 0.8″

7. The catheter of claim 1 wherein distal portion of the third tube forms the delivery tip.

8. The catheter of claim 1 wherein the delivery assembly is atraumatic and having
a. a smooth exterior surface,
b. a distal portion constructed of flexible material; and
c. the metal tube terminating before the distal portion of the delivery assembly.

9. The catheter of claim 1 wherein;
a. proximate end of the metal tube is connected to the handle hub providing proximate end of the inner lumen with an ability to be in fluid and suction communication with the irrigation fluid supply and the vacuum device through the handle hub and the control device; and
b. proximate end of the third tube is connected to the handle hub providing proximate end of the outer lumen with an ability to be in liquid communication with the container containing a treatment composition through the handle hub and the connector.

10. The catheter of claim 1 wherein the control device includes lockable features that removably connect the control device to both an aspiration line of the vacuum device and an irrigation line of the irrigation fluid supply, providing the inner lumen with bi-directional fluid flow for irrigation and aspiration without having to disconnect or reconnect the aspiration line and the irrigation line.

11. The catheter of claim 1 wherein the first tube and the second tube are constructed out of nylon; and the third tube, the fourth tube, and the delivery tip are all constructed out of thermoplastic material.

12. The catheter of claim 1 wherein the connector is comprised of Luer-lock fittings and the container containing the treatment composition is a syringe.

13. The catheter of claim 1 wherein the delivery nozzles are created by cuts into the third tube in a pattern of rows with (i) each of the rows is rotationally set apart from each other in a predetermined amount and (ii) each of the delivery nozzles is located in an offset fashion from the delivery nozzles located in the adjacent row.

14. The catheter of claim 13 wherein each of the delivery nozzles including a horizontal cut and a vertical cut that are perpendicular to each other.

15. The catheter of claim 14 wherein are the horizontal cut and the vertical cut are centered relative to each other.

16. The catheter of claim 1 wherein the treatment composition delivered by each of the delivery nozzles is in the form of small liquid droplets range from 5 μm to about 125 μm per droplet.

17. A multi-functional catheter comprising: a handle hub, a first tube, a second tube, a control device, a connector, and a delivery assembly comprising a third tube having a third lumen and delivery nozzles, a metal tube having a metal lumen, a fourth tube having a fourth lumen, and a delivery tip having a tip lumen; wherein
a. the delivery assembly includes a double lumen structure with an outer lumen and an inner lumen that is coaxial with the outer lumen and located within the outer lumen;
b. volume of the outer lumen is less than volume of the inner lumen;
c. the metal tube and the fourth tube are located within the third lumen and the outer lumen is formed by space remaining in the third lumen not occupied by the metal tube, the metal lumen, the fourth tube and the fourth lumen;
d. the inner lumen is comprised of at least a portion of the metal lumen, at least a portion of the fourth tube, and at least a portion of the tip lumen;
e. distal end of the first tube is connected, to the handle hub and proximate end of the first tube is connected to the connector; the connector is removably connected to a container containing a treatment composition;
f. distal end of the second tube is connected to the handle hub and proximate end of the second tube is connected to the control device;
g. the control device is removably connected to a vacuum device and an irrigation fluid supply;
h. the delivery assembly is connected to the handle hub;
i. the control device controls irrigation and aspiration by the inner lumen of the delivery assembly when the control device is connected to the vacuum device and the irrigation fluid supply;
j. the delivery nozzles are in communication with the outer lumen so when the connector is connected to the container, the delivery nozzles can deliver the treatment composition in a generally uniform distribution pattern onto a treatment site;
k. the delivery nozzles are located in distal portion of the third tube and constructed to assist in delivering the treatment composition in a generally uniform distribution pattern onto the treatment site;
l. the delivery assembly includes a central axis;
m. the delivery assembly has a first assembly section, a second assembly section, a third assembly section, and a fourth assembly section;
n. each of the first assembly section, the second assembly section, and the third assembly section includes the double lumen structure and the fourth assembly section includes the inner lumen;
o. the outer lumen extends along the central axis from proximate end of the delivery assembly to distal end of the third assembly section;
p. in the first assembly section, the third tube and the metal tube form the double lumen structure;
q. in the second assembly section, the third tube, the metal tube, and the fourth tube form the double lumen structure;
r. in the second assembly section, the metal tube is connected to the fourth tube allowing the metal lumen to be in fluid and suction communication with the fourth lumen;
s. in the third assembly section, the third tube and the fourth tube form the double lumen structure and the inner lumen is the fourth lumen;
t. the fourth tube is connected to the delivery tip allowing the fourth lumen to be in fluid and suction communication with the tip lumen;
u. the fourth assembly section includes the delivery tip and the tip lumen is the inner lumen;
v. combined length of the third assembly section and the fourth assembly section is less than length of the first assembly section;
w. proximate end of the metal tube is connected to the handle huh providing proximate end of the inner lumen with an ability to be in fluid and suction communication with the irrigation fluid supply and the vacuum device through the handle hub and the control device;
x. proximate end of the third tube is connected to the handle, hub providing proximate end of the outer lumen with an ability to be in liquid communication with the container containing a treatment composition through the handle hub and the connector; and
y. the delivery nozzles are created by cuts into the third tube in a pattern of rows with (i) each of the rows is rotationally set apart from each other in a predetermined amount and (ii) each of the delivery nozzles is located in an offset fashion from the delivery nozzles located in the adjacent row.

18. The catheter of claim 17 wherein distal portion of the third tube forms the delivery tip.

19. The catheter of claim 17 wherein in the second assembly section, the inner lumen is the metal lumen.

20. A method of using a multifunctional catheter to irrigate, aspirate, and deliver a composition to a treatment site comprising:
i. providing a multifunctional catheter comprising: a handle hub, a first tube, a second tube, a control device, a connector, and a delivery assembly comprising a third tube having a third lumen and delivery nozzles, a metal tube having a metal lumen, a fourth tube having a fourth lumen, and a delivery tip having a tip lumen: wherein
a. the delivery assembly includes a double lumen structure with an outer lumen and an inner lumen that is coaxial with the outer lumen and located within the outer lumen;
b. volume of the outer lumen is less than volume of the inner lumen;
c. the metal tube and the fourth tube are located within the third lumen and the outer lumen is formed by space remaining in the third lumen not occupied by the metal tube, the metal lumen, the fourth tube and the fourth lumen;
d. the inner lumen is comprised of at least a portion of the metal lumen, at least a portion of the fourth tube, and at least a portion of the tip lumen;
e. distal end of the first tube is connected to the handle hub and proximate end of the first tube is connected to the connector, the connector is removably connected to a container containing a treatment composition;
f. distal end of the second tube is connected to the handle hub and proximate end of the second tube is connected to the control device;
g. the control device is removably connected to a vacuum device and an irrigation fluid supply;
h. the delivery assembly is connected to the handle hub;
i. the control device controls irrigation and aspiration by the inner lumen of the delivery assembly when the control device is connected to the vacuum device and the irrigation fluid supply;
j. the delivery nozzles are in communication with the outer lumen so when the connector is connected to the container, the delivery nozzles can deliver the treatment composition in a generally uniform distribution pattern onto a treatment site; and
k. the delivery nozzles are located in distal portion of the third tube and constructed to assist in delivering the treatment composition in a generally uniform distribution pattern onto the treatment site;
ii. connecting the control device to an aspiration line of the vacuum device;
iii. connecting the control device to an irrigation line of the irrigation fluid supply;
iv. inserting the delivery assembly into a cavity until distal portion of the delivery assembly is located at a predetermined location near a treatment site;
v. irrigating: the treatment site using the catheter wherein the control device controls amount of irrigation fluid being delivered by the delivery assembly out of the delivery tip;
vi. aspirating the treatment site to remove waste material using the catheter wherein the control device controls amount of suction being delivered by the delivery assembly out of the delivery tip;
vii. applying the treatment composition in a generally uniform distribution pattern onto the treatment site using the catheter when the connector is connected to the container and the delivery nozzles are in liquid communication with the container; and
viii. removing the delivery assembly from the cavity.
